(19) United States
(12) Patent Application Publication
(10) Pub. No.: US 2024/0416060 A1
(43) Pub. Date: Dec. 19, 2024

(54) SYSTEM AND METHODS FOR DYNAMICALLY CONTROLLING OPERATION OF A MECHANICAL VENTILATOR FOR AUTOMATIC ADJUSTMENT OF ONE OR MORE SETTINGS BASED ON PATIENT BIOMETRIC DATA

(71) Applicant: Breas Medical AB, Molnlycke (SE)

(72) Inventors: William A. Truschel, Oakmont, PA (US); Harout Rafi Stepanian, Molnlycke (SE)

(73) Assignee: Breas Medical AB, Molnlycke (SE)

(21) Appl. No.: 18/742,760

(22) Filed: Jun. 13, 2024

Related U.S. Application Data
(60) Provisional application No. 63/521,579, filed on Jun. 16, 2023.

(51) Int. Cl. A61M 16/00; G16H 20/40; G16H 40/63; G16H 50/30

(52) U.S. Cl. CPC A61M 16/024; G16H 20/40; G16H 40/63; G16H 50/30; A61M 2202/0007; A61M 2202/0208; A61M 2205/3553; A61M 2205/502; A61M 2205/52; A61M 2230/005; A61M 2230/04; A61M 2230/20; A61M 2230/42; A61M 2230/46

(57) ABSTRACT

A system is provided for dynamically controlling operation of a ventilator for automatic adjustment of one or more operational settings based on patient biometric data. A ventilator prescription includes initial operational settings, minimum and maximum operational boundaries for each operational setting, a health score formulae for determining a current health score based on patient biometrics, an interval for evaluating current operational settings, and a selected optimization protocol for systematically trialing one of the operational settings to optimize the current health score. The ventilator starts with the initial configuration, monitors patient biometrics, periodically determines a current health score, systematically trials new operational settings and for each trialed setting that improves the Health Score resets the current configuration to the trialed confirmation (hill climbing methodology).

### CROSS-REFERENCE TO RELATED APPLICATIONS

This application claims the benefit of U.S. Provisional Patent Application Ser. No. 63/521,579 filed Jun. 16, 2023, the entire contents of which are incorporated herein by reference.

### BACKGROUND OF THE DISCLOSURE

### (1) Field of the Invention

The instant invention relates to mechanical ventilator systems, and more particularly to dynamic control of a mechanical ventilator which is not based on algorithmic analysis.

### (2) Description of Related Art

The state of the art in the practice of mechanical ventilation is performed under close supervision of a medical care professional in the intensive care unit (ICU). In the ICU, the patient is closely and continuously monitored with external biometric sensors, and the ventilator's alarm system. Any alert provokes an immediate response by the medical professional nearby and that caregiver can assess the patient, make any necessary changes to the therapy or therapy settings and watch, and wait for a positive response and healthy outcome for the patient before a medical emergency develops. Even in the absence of alerts, ICU caregivers are ordered to check the patient regularly such as every 4 hours, q4h, twice daily, bid, or even more frequently such that when the patient's condition changes care is appropriately adjusted timelier.

In contrast, when a patient is diagnosed with chronic respiratory insufficiency or chronic respiratory failure, the patient is often sent home with a mechanical ventilator on a fixed prescription. Although biometric sensors and the ventilator alarm system is still active in the home, it is common that no medical professional is nearby to change therapy or change the therapy settings to invoke or initiate a more effective care regimen for the patient. Follow up appointments are typically 6 to 8 weeks after discharge from the care of the intensivists in a hospital or clinic and regularly every 6 to 12 months after the first follow up.

Recent developments in connected care, allow a caregiver to review a patient remotely with a digital dashboard, but this review is often sporadic and spaced days, weeks, or months apart. When we consider that the solution involves allowing the clinician to change the settings for a critical patient, we incur a higher risk, and these remote unsupervised changes are discouraged. In fact, the message from the global regulatory bodies to allow a change to therapy for critical patients when the caregiver is remote is, “don't do it.”

Furthermore, the remote settings solution is not scalable as it requires the constant attention of expert manpower to monitor a large population of homecare patients within a health care system. Due to these challenges, it is expected that patients who need critical attention to a respiratory abnormality will not thrive under the current state of the art.

Due to the nature of the respiratory disease of the patient that may be considered progressive or in the category of a relapsing/remitting type from critical to stable at unknown frequency, it is often likely that therapy setting changes will lag behind the needs of the patient when the intervals of remote monitoring and patient follow-ups are considered.

### SUMMARY OF THE DISCLOSURE

The present disclosure relates to a problem associated with titrating a setting to meet the patient's needs and it addresses the need to regularly update the titration as the patient's needs change. As the patient's disease progresses or instances when the patient relapses into an acute situation or when the patient experiences a temporary change in physiology related to body position, immunological overload, infection, hypersecretion in the airway or other condition, it is often necessary to make a permanent or temporary modification to the prescription to improve the patient's respiration. The invention allows the clinician to program the device to update a prescription automatically by allowing the machine to continually trial and choose modified prescriptions that improve the clinician patient's measureable condition.

The process of initially titrating a prescription is often laborious and a burden to skilled personnel. Updating a prescription is also laborious and often ill-timed because patients with chronic disease in the home are often physically and temporally separate from their physicians. Updates are only prescribed during a follow up appointment and only after the laborious task of reviewing the patient's history made visible by downloading stored biometric data. This invention improves upon the state of the art by allowing the machine to continuously trial modifications to the prescription to determine the lowest configuration therapy that provides the maximum benefit to the patient condition. This will become clear by illustrating a new process by which the clinician can program a titration strategy into the prescription of one or more settings in the prescription mode.

Titration of a setting is done manually by starting with a typical prescription for the patient's demographics and diagnosis and then evaluating and reevaluating the patient as titration steps are carried out until a condition is satisfied, a maximal safe dosage is administered, or more aggressive treatment provides no additional benefit at the risk of more serious side effects. An example of this is to titrate an expiratory pressure level for a restrictive patient resulting in the lowest possible static lung compliance. Different expiratory pressure levels are tried, inspiratory hold maneuvers are performed where static lung compliance measurements are taken. The lowest expiratory pressure that produces the greatest lung compliance is preferred to keep the lung in the linear range without distention. When the titration has been performed, it is assumed that this prescription for expiratory pressure or other parameters or settings examined will be suitable across time between doctor visits or remote data examinations, only during which a modification to the prescription may seem necessary. Furthermore, the titration may be temporarily substandard when the patient undergoes a temporary change in physiology due to an infection, progressive disease, immunological overload or when the patient's physical position changes that may require an increase in expiratory pressure (PEEP) in order to maintain a suitable lung compliance.

Manufactures have begun to address these issues to improve home mechanical ventilation for unstable patients in two ways. They have developed sophisticated auto-titrating algorithms such as VAPS modes, which stands for Volume Assured Pressure Support. In VAPS systems, the pressure support will increase or decrease as tidal volume decreases or increases respectively. The VAPS system can respond quickly to changes in respiratory drive, airway obstruction or lung or chest wall compliance to normalize the depth of breathing for the patient. Normal depth of breathing is often specified by a target typically related to the height and gender of the patient. Systems such as iVAPS developed by Resmed (Sydney, Australia) go one step further and adjust pressure support based on estimated alveolar ventilation and consider the patient's breathing rate to normalize the estimated minute ventilation of the patient towards a target value based on patient demographics. These systems are non-ideal in two ways. First, they do not consider the changing metabolic needs of the patient that require deeper breathing or higher ventilation at times. Secondly, they require a priori knowledge of what is normal or optimal for the patient when setting the target tidal volume or minute ventilation.

Although VAPS modes have been widely used and established, these modes lack consistency between manufacturers and often operate in an opaque manner that the medical professional does not fully understand. For example, the Breas (Mölnlycke, Sweden) VAPS version is called (Target Volume) TgV. The TgV algorithm adjusts pressure support (PS) by 0.5 mbar in the direction opposite of the difference between the measured tidal volume on the last breath vs. the target tidal volume. TgV does not adjust PS when the measured volume is within 5% of the target volume. Alternatively, the VAPS from Philips Respironics, (PRI, Pittsburgh Pennsylvania) adjusts PS accordingly in the opposite direction of the difference between the measured tidal volume on the previous breath based on the estimated dynamic compliance of the lung. PS is adjusted by Resmed (Sydney Australia) at rates far greater than both Breas and Philips according to the proportional difference between the measured tidal volume and breath rate product and the target minute ventilation. The lack of transparency and understanding of these algorithms can discourage caregivers who may be hesitant to prescribe a therapy regimen that they don't fully understand when they remain fully responsible for the health and care of their patients.

Programmable Ventilation is a process where the clinical user may prescribe a range of values for a particular parameter or operational setting within the prescription to benefit the patient's changing needs without any a priori knowledge of the optimal or normal condition of the patient. In this invention, no a priori knowledge of what is best for the patient is necessary because the device will continually attempt to achieve the optimal settings to maximize a quantifiable measure of the patient's respiration (referred to herein as a “Health Score”) even when the needs or the morbidity of the patient is continually changing.

Secondly, this invention overcomes the concern that existing machine's designed with secret or opaque algorithms (Such as VAPS or iVAPS) may harm a patient. Here, the clinician has complete control and transparency related to the operation of all actions related to the titration including when, why, to what extent and how each parameter will change.

Auto set parameters in this invention include prescription parameters that could be adjusted within a range such as back up rate, rise time, inspiratory time or FiO₂. As a matter of example, the clinician may adjust the rise time parameter controlling the inflation rate of the pressure to maximize the minute ventilation in a pressure control mode. In this example, the rise time will be constrained within the programmed range of rise times that are both pre-programmed by the clinician and within the validated safety and performance limits of the device.

The invention intends to overcome the issues of inappropriate response time incurred due to remote monitoring and more specifically no response when caregivers are absent, or the patient is unable to intervene on his own. The clinician ultimately knowing what is best for an individual or class of patients and has full authority over the intervention actions. No hidden algorithm is contained within the device to automatically make a clinical assessment of the patient and the device does not decide what any appropriate response could be. The device will only operate according to the direction of the programming clinician and there is full transparency of how, when, and why a setting will change. The method described here can be accomplished without using closed loop control as defined by MDR classification rules explicitly mentioned in MDCG 2021-24 under Rule 12.

The key distinction with “programmable ventilation” as described herein, is that control of the ventilator's settings is solely controlled by the signals monitored and displayed by the device without providing any “diagnostic function” for analysis of the patient's biological condition and physiological state. For example, an increase in applied expiratory pressure is determined necessary when the increase in expiratory pressure during a trial results in a improved static lung compliance. In this example, the device makes no diagnostic determination that the patient is experiencing atelectasis or restrictive lung disease, the control is simply modified based on the static lung compliance sensor's measurement. The device will not manage the patient according to any incorporated diagnostic algorithm, rather it will adjust one or more prescription parameters to improve the static lung compliance or any other respiratory measurements that the clinician has chosen to be related to the health or the morbidity of the patient.

The system adjusting the parameter or parameters shall be constrained to approved ranges ascribed for the device and the class of patient. In other words, the clinician cannot intervene with a prescription that is not already allowed under the device's intended use or qualified range of settings and the machine shall not be capable of performing a trial outside of the clinician's chosen limits. The procedures and techniques including the stability analysis, verification, and validation of the basic device will ensure compliance with the applicable safety standards and that all associated safety risks have been managed as far as possible in the design.

According to exemplary embodiments, a system and method are provided for dynamically controlling operation of a mechanical ventilator for automatic adjustment of one or more operational settings based on patient biometric data. A ventilator prescription includes a plurality of initial operational settings, minimum and maximum operational boundaries corresponding to each of the plurality of operational settings, a health score formula, an interval period for reliably evaluating current operational settings of the ventilator system applied to the patient's response to the treatment, assigning a series of current health scores corresponding to the various trialed modifications to the operational parameters using formulae by which the ventilator determines a current health score according to one or more biometric variables corresponding to the target person; and a selected optimization method by which the ventilator systematically trials a plurality of operational settings within the boundaries to learn the optimal health score achievable by applying configuration settings between the assigned boundaries. The ventilator system operates according to the initial configuration, monitors patient biometric variables, assigns a health score related to the initial configuration, continually modifies the configuration within the boundaries, monitors patient biometric variables, assigning a health score related to the new configuration, tracks in memory the health scores for different and current operational settings, continually chooses the configuration settings that encourage the optimal health score, and proceeds to further modify the operational settings so long as the health score is improving with each modification. The systematic method of generating modifications to the initial or current operational settings (increase or decrease) in setting (dosage) are defined by the regular ascent of the health score towards the optimal health score produced by the lowest dosage. This method is commonly referred to as a hill climbing algorithm. It reconfigures the ventilator system according to the trialed modification that benefits the patient (increases health score), as if the machine is blindly climbing a hill using the altimeter measurements of a health score. Any configuration that improves the health score will immediately become the current configuration, thus providing the patient with beneficial updates to the configuration continually.

The health score is based on a plurality of biometric sensors and the correlation of each sensor measurement to patient health. For example, the correlation between CO₂ and health is negative for hypercapnic patients because lower CO₂ is “healthier”, SpO₂ is positively correlated to health for hypoxemic patients because higher SpO₂ is “healthier”, respiratory rate is negatively correlated to tachypneic patients because lower respiratory rate is “healthier” for these patients. The clinician can choose one or more biosensors to appropriately score the health of the patient and the machine applies a formula based upon the chosen biomarkers and correlation coefficients. The invention shall adjust the configuration parameters within the operational boundaries to maximize this chosen health score with a hill climbing trial system.

In some embodiments, the initial configuration may comprise one or more predetermined operational settings and/or operating modes which include but are not limited to the following: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

The operational settings associated with these modes include but are not limited to: Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (AP). In many instances the base mode and intervention mode may be the same, but the intervention mode may include one of more differences in operation parameters that are all pre-determined by the prescribing clinician to affect positive outcomes when the patient is in need.

In some embodiments, the biometric variables may include, but are not limited to the following: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

In some embodiments, the biometric variables may further include calculated biomarkers such as Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (ΔP), Mechanical energy, Mechanical power and intensity, and Pressure-time product per minute (PTP).

In some embodiments, the optimization formulae identifies modifications of two or more of operational settings to optimize the current health score.

In some embodiments, the optimization formulae identifies a plateau condition of decreasing improvement to the current health score and maintains current operational settings in a steady state condition.

While embodiments of the invention have been described as having the features recited, it is understood that various combinations of such features are also encompassed by particular embodiments of the invention and that the scope of the invention is limited by the claims and not the description.

### BRIEF DESCRIPTION OF THE DRAWINGS

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1 illustrates a ventilator system;

FIG. 2 illustrates lungs and the inspiration and expiration flows in and out of the lungs;

FIG. 3 illustrates a schematic of a programmable physician managed auto titration system in accordance with the teaching of the present invention;

FIG. 4 is a chart of common ventilator operating modes and associated parameters set for each mode along with common values and units.

FIG. 5a illustrates the most common form of the hormetic dose-response curve depicting low-dose stimulatory and high-dose inhibitory responses, the β- or inverted U-shaped curve where endpoints displaying this curve include growth, fecundity and longevity.

FIG. 5b illustrates the hormetic dose-response curve depicting low-dose reduction and high-dose enhancement of adverse effects where endpoints displaying this curve include carcinogenesis, mutagenesis and disease incidence (FIGS. 5a and 5b sourced from Nonlinearity Biol. Toxicol. Med. 2003 July; 1 (3): 319-343);

FIGS. 6a and 6b illustrate representative examples of dose-response relationships that are consistent with a hormetic hypotheses in FIGS. 5a and 5b (FIGS. 6a and 6b sourced from Nonlinearity Biol. Toxicol. Med. 2003 July; 1 (3): 319-343);

FIG. 7 illustrates a fundamental dosage response curve with three distinct regions;

FIG. 8 illustrates changes to the fundamental curve that may develop and manifest in a patient with progressive disease receiving treatment;

FIG. 9 illustrates a different response curves for a disease that varies between varying severity of relapses and remittance curves;

FIG. 10 illustrates in flow chart form a basic technique to continually change the dosage in the direction of increasing vitality score where the dosage will do “hill climbing” on a smooth response curve using the depicted flow patterns;

FIG. 11 illustrates a fundamental response curve resulting from the hill climbing flow method as illustrated in FIG. 10;

FIG. 12 illustrates how the machine reacts if there is a relapse, followed by a remittance, showing that the hill climbing technique follows the response curve during relapse and remittance, providing higher dosages during a relapse and lower dosages during a remittance;

FIG. 13 demonstrates the performance with a progressive disease where the dosage gradually increases as the disease severity progresses;

FIG. 14 shows the response of the machine to a hormesis response with and ideal dosage of 50 (μ=50) and a low starting dosage;

FIG. 15 shows the response to a hormesis response with an ideal dosage (μ=50) but with a high starting dosage;

FIG. 16 shows the performance of the stochastic hill climber performing on a complex dose/response, starting at different dosages within range on different days;

FIG. 17 illustrates machine response for a two setting titration wherein following the steepest ascent, the machine finds the optimal settings to reduce symptoms on this complex surface;

FIG. 18 illustrates the circular search patterns that are routinely performed to find the direction of the steepest ascent where small changes are made to the current settings in a circular pattern, making measurements to determine the steepest ascent away from the current settings;

FIG. 19 illustrates a graphical user interface for setting of an operational value as a fixed value in a non-titrating state;

FIG. 20 illustrates a graphical user interface for setting of an operational value with an initial starting value and a range of operational values that the system can utilize during titration;

FIGS. 21A and 21B depict a chart illustrating basic operational settings for an auto-titrating functionality;

FIG. 22 illustrates a graphical user interface for selecting one or more biometric readings to evaluate the health of the patient at the given prescription; and

FIG. 23 illustrates a flow chart of system operation.

### DETAILED DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

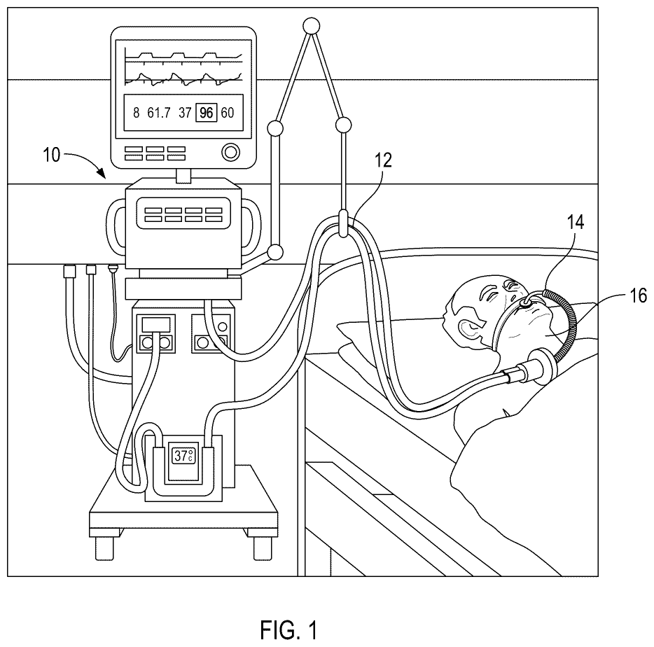

Referring to the drawing figures, FIG. 1 illustrates a basic ventilator system 10 that provides pressurized air through the tube 12 into an airway adaptor 14, such as a mask, to the user/patient 16. In some instances, a mask is not used, where the tube is directly fed into the trachea, such as a tracheostomy.

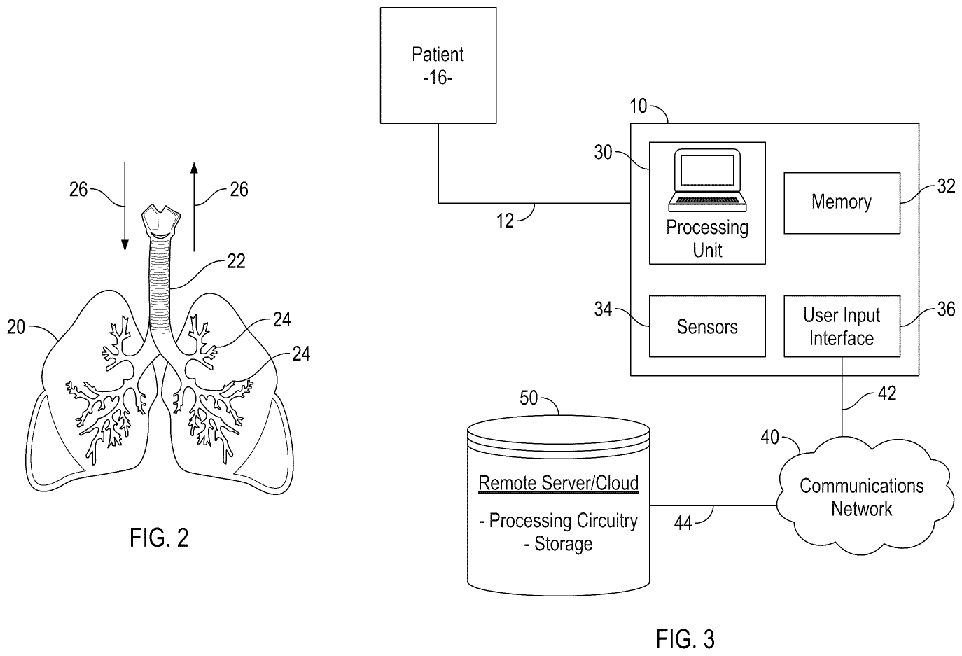

FIG. 2 illustrates lungs 20, including the trachea 22 and the bronchi of the lungs 24. The inspiration flow path 26 travels into the trachea 22 and into bronchi 24, whereas the expiration flow path 28 travels or flows out from the lungs 20 and bronchi 24 into and out of the trachea 22.

FIG. 3 illustrates a schematic of an auto-titrating mechanical ventilation system 100 that includes a ventilator system 10, which includes a processing unit 30 configured to receive input operating parameters (as set forth hereinbelow), via a user/patient input interface 36, including configuration settings in the form of a prescription, settings and parameters, as well as direct and processed sensor data captured by sensors 34 (machine based as well as external patient biometric sensors), to recall and place data into memory 32, and direct communications over a network 40 to remote server/cloud 50 that also includes processing circuitry and storage. Directed communications 42 can be made to and from the communications network, which can make directed communications 44 to and from the remote server/cloud 50. Cloud computing is generally understood in the art to mean the delivery of computing services-including servers, storage, databases, networking, software, analytics, and intelligence-over the Internet (“the cloud”) to offer faster innovation, flexible resources, and economies of scale.

Some definitions and abbreviations that will be helpful for this application including the following:

Inspiratory flow refers to the flow of air entering into and flowing towards the lungs.

Expiratory flow refers to the flow of air exiting the lungs and flowing towards the glottis.

Rise Time is the rate at which the pressure ramps up to the prescribed or determined pressure level. The pressure generally rises during inspiration. The rise time of pressure can affect the flow rate and in particular the inspiratory flow rate and in more particular the peak inspiratory flow.

Fall Time is the rate at which the pressure ramps down to a determined pressure level. The pressure generally decreases during expiration. The fall time of pressure can affect the flow rate and in particular the expiratory flow rate and in more particular the peak expiratory flow.

Prescribed Pressure or Pressure Dosage is the amount of pressure that the ventilator ramps up to during use of the ventilator and is usually prescribed by a medical provider. Units of pressure are usually in the form of cm H₂O or centimeters of water column. Prescribed pressures generally range from 5-25 cm H₂O, and generally do not exceed 30 cm H₂O.

Mechanical ventilators are operational in several different modes. The most common modes of mechanical ventilation include but are not limited to: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

Volume control modes are generally favored for ventilation control, while pressure control modes are favored for assisted or spontaneously breathing patients. Both types of modes have advantages and disadvantages that are mainly related to the flow and pressure patterns of gas delivery.

Mechanical ventilators have many parameters that clinicians can adjust to treat patient, including but not limited to:

| Parameter | Value | Units |
|---|---|---|
| Peak Inspiratory Pressure | 0-99 | cm H2O, mbar, hPa |
| Peak Inspiratory Flow | 0-200 | L/min |
| Inhaled Tidal Volume | 0-2000 | ml |
| Exhaled Tidal Volume | 0-2000 | ml |
| Respiratory Rate | 0-120 | BPM |
| Minute Volume | 0-40 | L/min |
| % Spontaneous Trigger | 0-100 | % |
| Asynchrony Index | 0-100 | % |
| Lung Compliance | 1-200 | ml/cm H2O, ml/mbar, ml/hPa |
| Static Lung Compliance** | 1-200 | ml/cm H2O, ml/mbar, ml/hPa |
| Static Lung Resistance** | 1-200 | cmH2O/Lps, mbar/Lps, hPa, Lps |
| Plateau Pressure** | 0-99 | cm H2O, mbar, hPa |
| Alveolar Pressure | 0-99 | cm H2O, mbar, hPa |
| Expiratory Resistance | 1-200 | cmH2O/Lps, mbar/Lps, hPa, Lps |
| Inspiratory Resistance | 1-200 | cmH2O/Lps, mbar/Lps, hPa, Lps |
| iPEEP | 0-40 | cm H2O, mbar, hPa |
| FiO2 | 21-100 | % |
| SpO2 | 5-100 | % |
| Heart Rate | 18-321 | BPM |
| ETCO2 | 5-150 | mmHg |
| TcCO2 | 5-150 | mmHg |
| Peak Expiratory Flow | 0-200 | L/min |
| I:E Ratio | 2:1:1:99 | |
| Expiratory Time | 1-60 | sec |
| RSBI | 8-999 | Breaths/min/L |
| Vt/kg IBW | 1-30 | cc/KgIBW |
| Flow Bias Ratio* | 10:1:1:10 | Insp:Exp |
| Average Leak | 0-200 | L/min |
| Leak Ratio to Normal | 0.0:10 | None |
| AHI (Apnea Hypopnea Idx) | 0-300 | per hour |
| Expiratory Time Constant | 0.1-10 | sec |
| Resp Rate-Oxygenation (ROX) | 0-99 | points |

Abbreviations associated with the above are set forth below.

| Parameter Name | Abbreviation |
|---|---|
| Peak Inspiratory Pressure | PIP |
| Peak Inspiratory Flow | PIF |
| Tidal Volume | Vt |
| Respiratory Rate | RR |
| Minute Volume | MVexp |
| % Spontaneous Trigger | % Spon Trigger |
| Asynchrony Index | Asynch Idx |
| Lung Compliance | Clung |
| Static Lung Compliance** | Cstat |
| Static Lung Resistance** | Rstat |
| Plateau Pressure** | Pplat |
| Alveolar Pressure | Palv |
| Expiratory Resistance | Rexp |
| Inspiratory Resistance | Rinsp |
| Intrinsic PEEP | iPEEP |
| Fraction of Inhaled O2 | FiO2 |
| Saturation of Peripheral O2 | SpO2 |
| Heart Rate | HR |
| End Tidal Carbon Dioxide | ETCO2 |
| Transcutaneous Carbon Dioxide | TcCO2 |
| Peak Expiratory Flow | PEF |
| Inspiratory Expiration Ratio | I:E Ratio |
| Rapid Shallow Breathing Index | RSBI |
| Tidal Volume per Ideal Body Weight | Vt/kg IBW |
| Flow Bias Ratio* | PEF/PIF Ratio |
| Average Leak | Ave Leak |
| Leak Ratio to Normal | Leak Ratio |
| Apnea Hypopnea Index | AHI |
| Expiratory Time Constant | TCexp |
| Inhaled Tidal Volume | Vti |
| Exhaled Tidal Volume | Vte |
| Resp Rate-Oxygenation (ROX) | ROX |

Additional definitions are also set forth below.

Positive end-expiratory pressure (PEEP)

Inspiratory airflow (V′)

Percentage of inspired oxygen (FiO₂): Normal is 35-50%, but can be increased to 75-100% if needed

Air pressure (SetP)

Peak inspiratory pressure (PIP): The sum of PEEP and SetP

Peak pressure

Inspiratory time: Inspiratory Time is the length of time during which the ventilator delivers the inspiratory pressure.

Inspiratory-to-expiratory ratio

Time of pause

Trigger sensitivity

Support pressure

Expiratory trigger sensitivity

Ventilators can also produce ventilator-derived parameters, i.e. biomarker calculators, which can be used to guide ventilatory strategies and detect problems with the ventilator or changes in the patient:

Intrinsic PEEP (PEEPi): The residual pressure when the expiratory phase isn't fully completed

Plateau pressure (Pplat): Equal to alveolar pressure when airflow is zero

Transpulmonary pressure

Transpulmonary driving pressure (AP)

Mechanical energy

Mechanical power and intensity

Pressure-time product per minute (PTP)

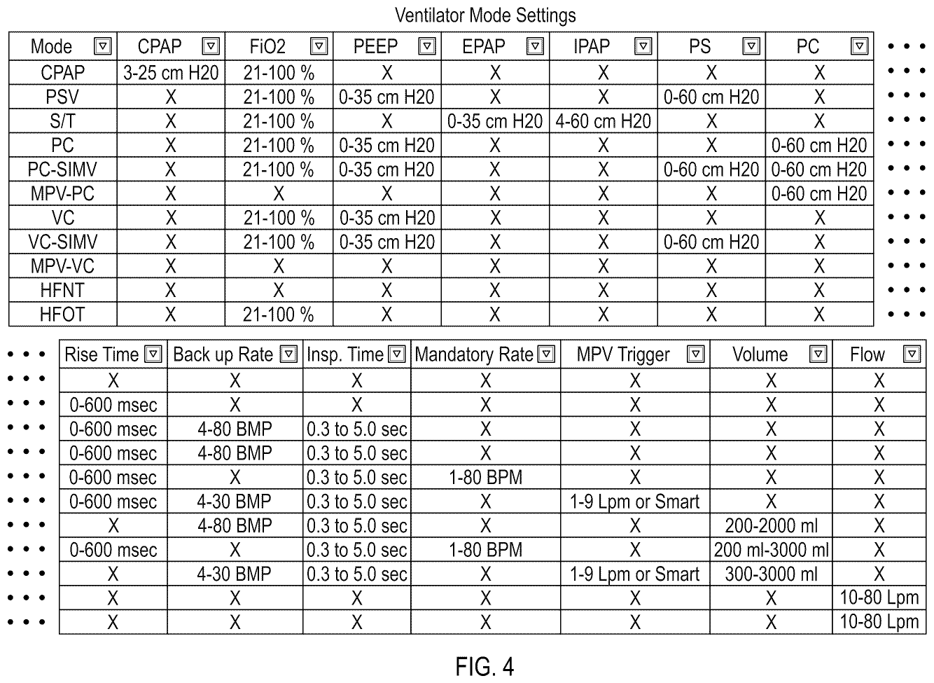

Finally, parameters (or settings) to be set depend on modes. An exemplary setting chart is set forth in FIG. 4, where the settings names are the column headers.

Patients on mechanical ventilation are usually monitored in an intensive care unit (ICU) and have monitors that measure several values related to respiration, including but not limited to: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

### Treatment Regimens

In general, chronic disease patients will fall into 4 categories. There are stable patients such as those with a spinal cord injury in which the lungs are healthy, but the respiratory drive is and will always be zero. These patients need little maintenance done to their prescription pressure dosage. Secondly there are patients with progressive disease and these patients need regular increases to their prescription dosage. Thirdly, there are patients with a disease that presents in a relapsing/remitting pattern and these patients need prescription updates at an irregular interval when the patient state switches between a relapse of variable intensity to a remittance that is asymptomatic or hyposymptomatic. Finally, there are patients who exhibit combinations of the previous 3 types.

We typically expect to treat these patients with a particular mode of ventilation but often one or more the prescriptions setting in this mode should likely be changed to meet the changing needs of the patient. We can demonstrate and control these changes to the prescription fully as a function of parameters that monitor the response of the patient to the value of the operational setting, here described as a dosage.

Much has been studied on the relationship between response and dosage and we shall consider this relationship as the basis for changing a patient prescription by assuming the response is an unknown objective function. The invention described here relates the chosen biometric data to a series of candidate dosages, but systematically trialing, experimenting and learning which dosages produce the optimal patient response (as defined by the monitored parameters selected for the individual patient that relate to his symptoms).

The literature suggests that dosage responses can take on two basic forms. One form referred to as the fundamental response is that of a sigmoid, where the lowest dosages are ineffective, until effectivity is reached at a minimum dosage. The sigmoid teaches that the response increases with dosages in the increasing effect range until a maximum effective dosage it trialed. The maximum effective dosage may be defined by a removal of any symptoms, as an inhibitor to any adverse conditions associated with the chronic disease or simply by detecting that the utility of the setting increases have reached a limit. (It's as good as it gets) Additional dosage beyond the maximum effective dosage has no additional benefit and can often be at higher risk for adverse side effects.

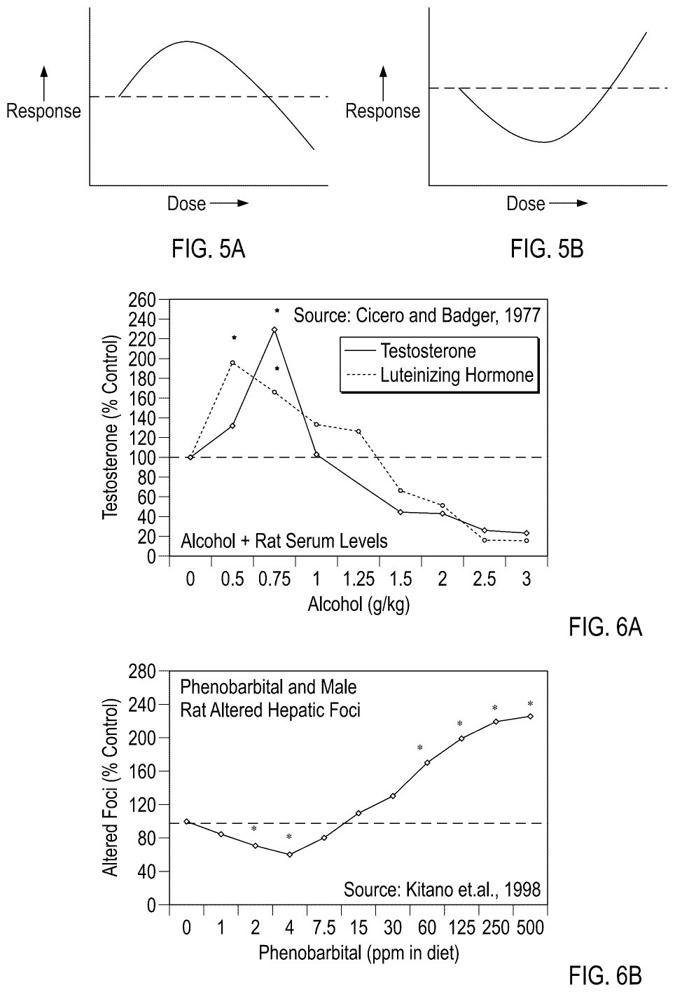

The other form of dosage response is that of an inverted U shape. This response is known as hormesis, and many drugs behave this way as illustrated in FIGS. 5a-5b and FIGS. 6a-6b. It has been argued that the U-shaped response is far more common than the fundamental response, but like the sigmoidal response, there is little to no effect at the lowest dosages. The response improves as the dosage increases until the maximal effect has been reached. In the U-shaped response, the response degrades with higher dosages above the optimal dosage.

With ventilator settings, it is expected that the patient response is expected to be like one of these two functions. (The fundamental response curve or the inverted U-shape) The ventilator must be expected to behave similarly well with either type of response curve.

For illustration, a four parameter logistic function has sometimes been used to represent the fundamental response/dosage curve,

$$f ( x ) = c + \frac{d - c}{1 + {exp}^{b ( log ( x ) - log ( e ) )}}$$

But here, we use the Gompertz function with only 3 parameters to represent the fundamental patient response to a variable dosage of a prescription. The Gompertz function is a sigmoidal function that predicts mortality rates in populations, but here we have repurposed it to illustrate a point.

The Gompertz function is a configurable function with 3 parameters that can generically approximate the fundamental potency relationship between medications, therapy settings on a device and pharmacological dosages.

This function is described with the function

$$f ( t ) = {ae}^{- {be}^{- ct}}$$

Where t is the time variable for mortality rates, but the equation was modified here to describe the effectiveness of a dosage (d)

With

$$eff ( d ) = {ae}^{- {be}^{- cd}}$$

Where
the maximum effect a drug can have is defined by the parameter, a,
the parameter effecting the minimum effective dosage is, b,
the parameter, c, sets the effectiveness change vs dosage in the increasing response range of a dosage.
e is Euler's number.

It is supposed that a logistic function, a hyperbolic tangent or any sigmoidal function with two plateaus and an increasing effect zone betwixt the plateaus can be assumed. In this method, the selection of the function is immaterial to the concept.

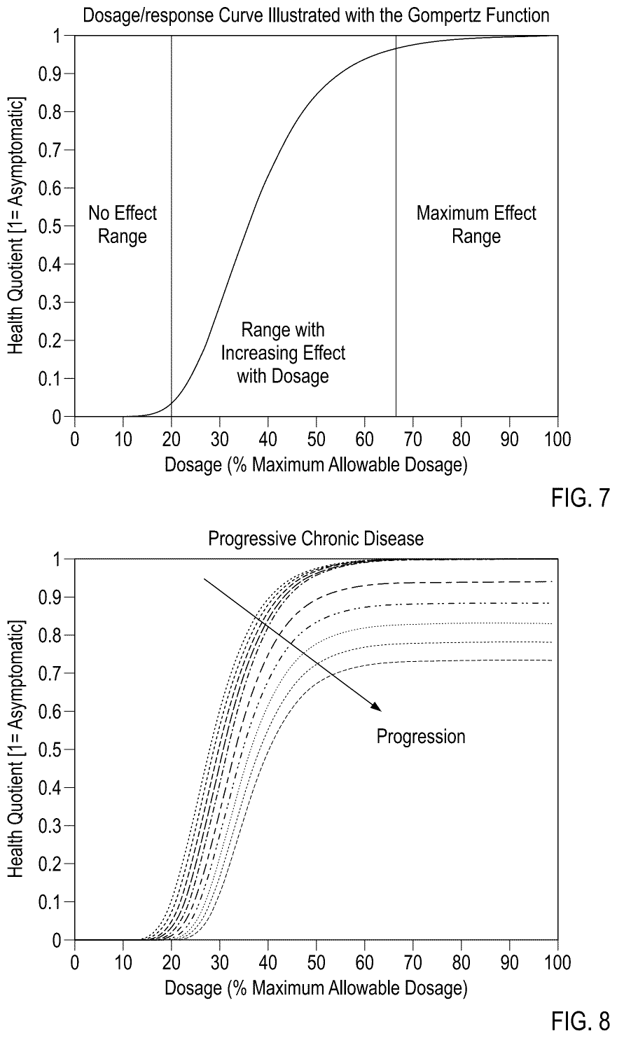

The fundamental response curve has three regions as illustrated in FIG. 7. With Health Quotient on the Y-axis and Dosage on the X-axis. The terms “Health Quotient”, “Health Score” and “Vitality Score” may be used interchangeably throughout the specification, figures and claims, with a “score” of 1 being asymptomatic (best case) and 0 being fully symptomatic (worst case).

What is important to realize when managing the dosage over time for a patient with chronic disease is that these curves change over time and that at each time, the algorithm should attempt to maximize the effectiveness within safe limits while minimizing the risk of side effects related to overdosing beyond the lowest maximum effective dosage.

The curves illustrated in FIG. 8 represent what changes to the fundamental curve may develop and manifest in a patient with progressive disease receiving treatment.

As the disease progresses, the patient may require a higher dosage. (for example, a higher pressure support to normalize his CO₂, but with further progression, normocapnia may not be achievable with any dosage and this is illustrated by the reduction in the plateau level with disease progression.)

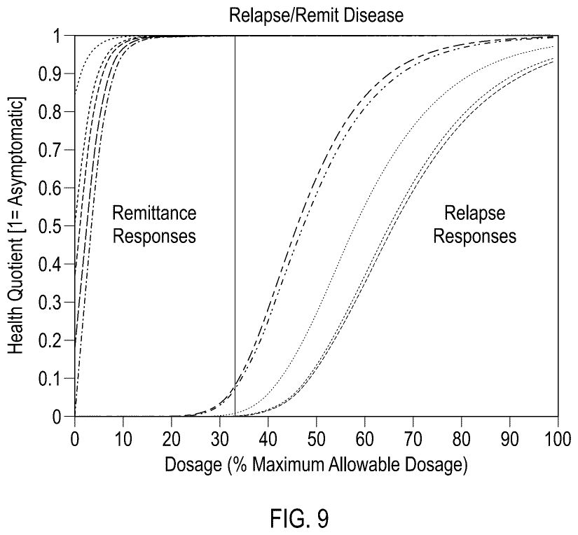

Referring to FIG. 9 there is illustrated representation of different response curves for a disease that varies between varying severity of relapses and remittance curves. During remittance, the patient is asymptomatic with little or no need for medication while the during a relapse, a higher dosage or varying effect is necessary to apply.

If a disease such as obstructive sleep apnea OSA is present, the relapse/remittance periods may change in just a few minutes with sleep stage, so it is important that the administration of dosage changes are geared toward the specific characteristics of the disease. (in the case of OSA, the dosage is CPAP level) It is also important that dosage changes occur at the appropriate interval for the nature of the disease.

### Health Score

In programmable systems, the Health Score, Vitality Score or Health Quotient, as previously mentioned, is defined by the clinician. The health score is a quantity that indicates a quantitative value from which intervention by dosage can improve. It is one measurement, or a combination of measurements made by the controlling ventilator, or the biometric sensors connected to the ventilator that are monitoring the patient while the auto-titrating algorithm is active. Consider the health score as a fitness function that represents a quantitative measure of the response of the patient to the treatment.

Relevant potential examples in the practice of mechanical ventilation are provided herein.

For purposes of illustration we include the following representative example.

### Example 1

A clinician wishes to treat a hypoxemic/hypercapnic patient with end stage COPD. The patient's tidal volume is titrated with a pressure support mode including VAPS, but it is also desired to control FiO₂. For this biometric, a pulse oximeter is connected to the patient for the measurement of SpO₂ and Heart Rate.

Here hypoxemia is a symptom of end stage COPD and treated with a greater partial pressure of inhaled oxygen. The symptomatic score of hypoxemia, will be derived from measurements of the pulse oximeter.

The machine knows that hypoxemia is diagnosed with increased HR (Negative Correlation to health related to tension of oxygen in the blood.)

The machine knows that hypoxemia is diagnosed with lower SpO₂. (Positive Correlation to the tension of oxygen in the blood)

Therefore the Symptomatic score is computed as

$$Health Score = \frac{Sp O 2}{HR}$$

The health score is determined from the signals chosen by the clinician to include the terms Heart Rate and SpO₂ and the machine will compute the score as the product of the positively correlated metrics divided by the product of the negatively correlated variables.

$$Health Score = \frac{\prod Positively Correlated Metrics}{\prod Negatively Correlated Metrics}$$

The health score should follow either a U-shape when the dosage/response curve is hormetic or a sigmoid when the dosage/response curve is fundamental.

It can be considered that it would be easy to modify the health score by flipping it and measuring the symptomatic score that should be minimized. We can refer to this score as a “Symptomatic Score”. A higher Symptomatic score will indicate a negative patient response to the dosage. The symptomatic score is simply the inverse of the health score.

$$Symptomatic Score = \frac{1}{Health Score}$$

### Methods of Finding the Optimal Setting by Measuring the Response of Trial Dosages

The methods used for finding the optimal settings involving changing the settings (trialing or titrating) different dosages in a programmed way and using repeated measurements at each dosage to determine which dosage provides the best response. The problem to be solved is for the machine to continually and efficiently adjust and provide the greatest achievable Health score or the minimal Symptomatic score. This technique of finding the minimum Symptomatic score or the maximum Health score applies equally to both forms of response curves (fundamental and U-shaped). Ascending most efficiently and avoiding plateaus that are non-optimal can be accomplished by methods described below.

### Basis Technique of Hill Climbing for Optimizing Vitality for Simple Dosage/Response Curves.

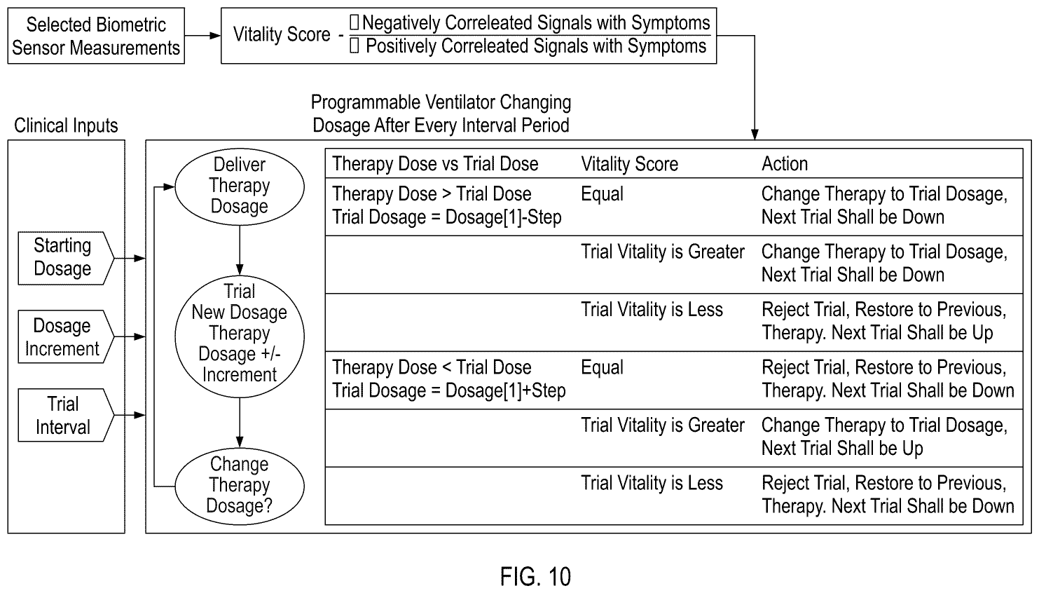

A basic technique to continually change the dosage in the direction of increasing vitality score in shown in FIG. 10. The dosage will do “hill climbing” on a smooth response curve using the flow chart illustrated therein.

The user provides the clinical inputs for the machine to adjust dosage. The dosage begins at the starting dosage in most cases, but occasionally, the dosage may change within the limits to increase the vitality score. The machine should not deliver a dosage that is above the maximum effective dosage to minimize the risk of adverse side effects from high dosages.

The dosage is changed at a frequency determined by the clinician's input for trial interval based on the expected response time of the patient to the therapy and the need to react to sudden changes in patient condition based on severity. This is done without exception unless the device fails to obtain valid measurements during the interval period and can't make a good assessment at the trial dosage.

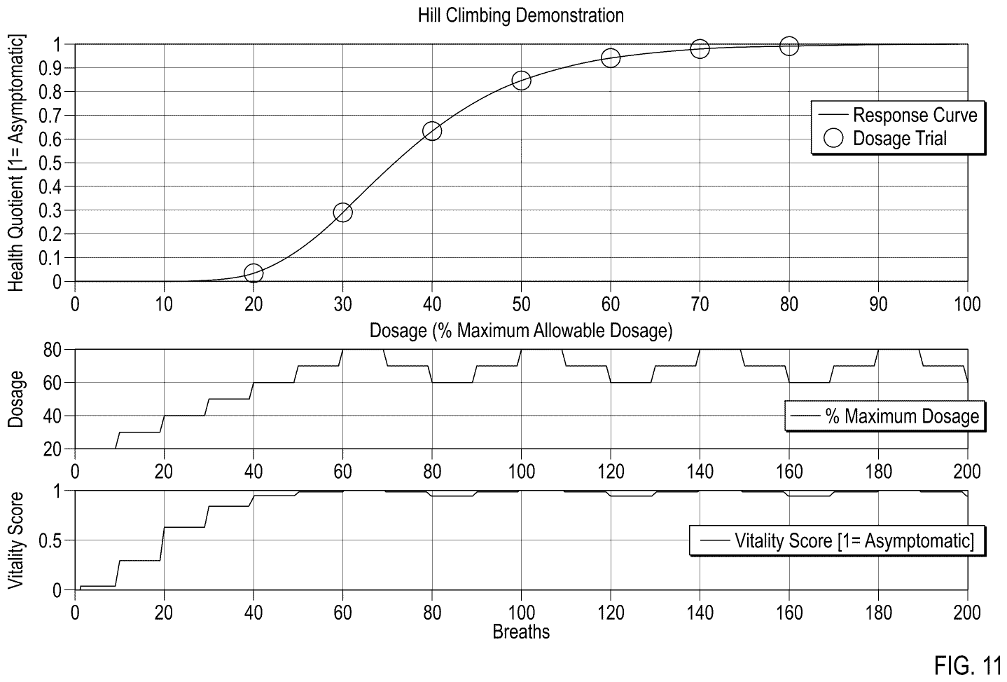

Applying this technique to a fundamental response curves results in the behavior illustrated in FIG. 11 In the illustrated example, the starting dosage is 20, the step dosage is 10, the interval is 10 breaths. The dosage climbs the hill until the dosage is 80 and then reverts and settles when there is no significant improvement in the response above a dosage of 80.

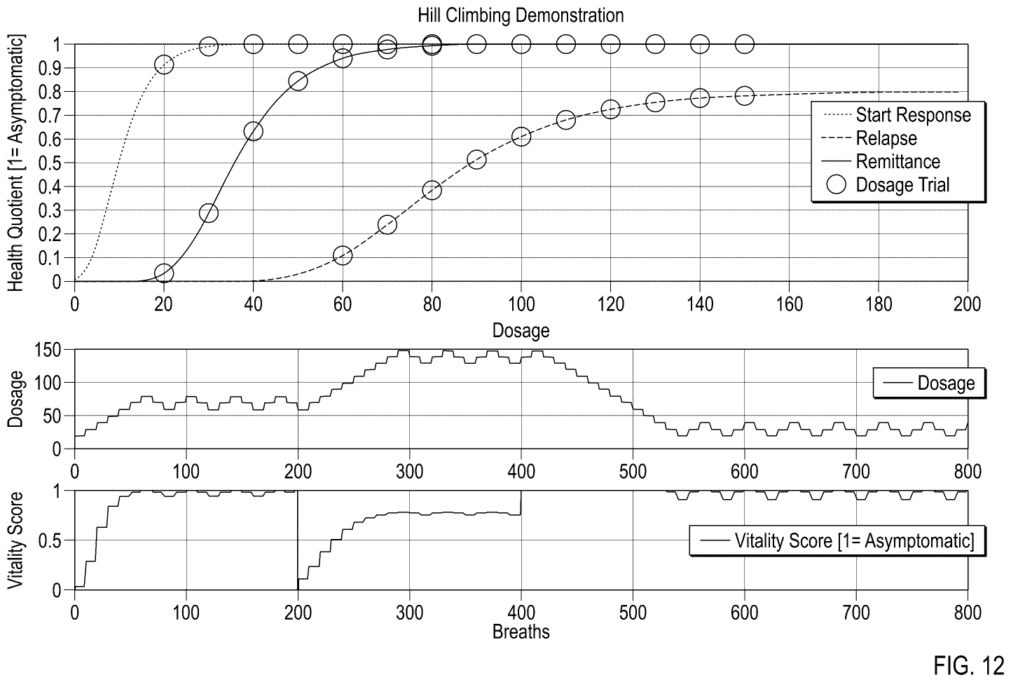

FIG. 12 illustrates how the machine reacts if there is a relapse, followed by a remittance. FIG. 12 shows the hill climbing technique follows the response curve during relapse and remittance. It provides higher dosages during a relapse and lower dosages during a remittance.

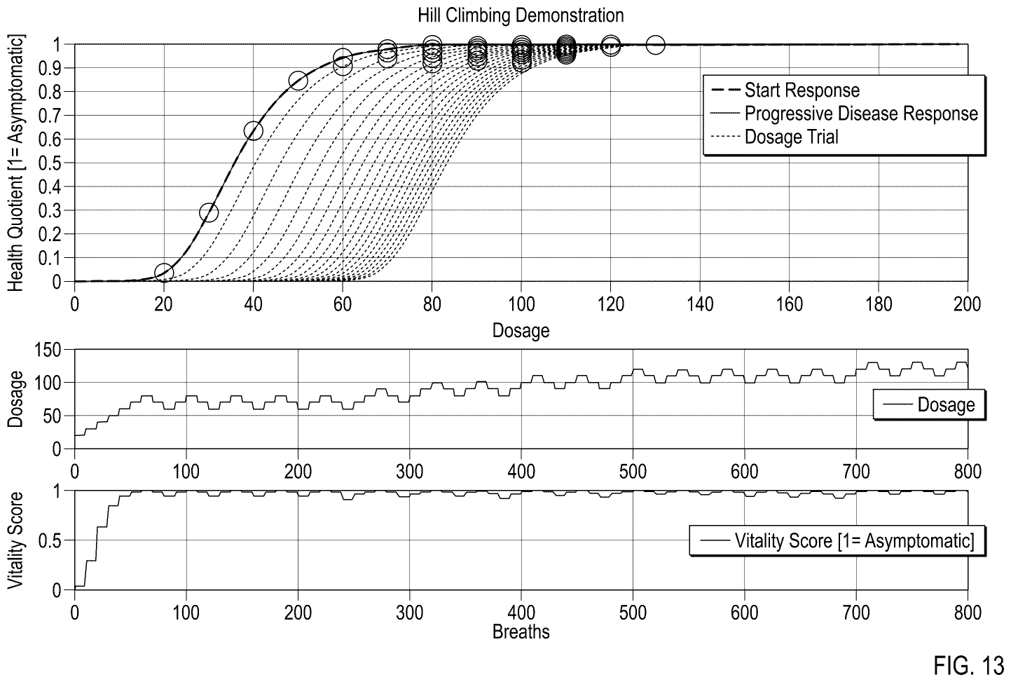

FIG. 13 demonstrates the performance with a progressive disease. Here, the dosage gradually increases as the disease severity progresses.

To demonstrate how this machine would function with a hormesis response (U shaped), we demonstrate Hormesis functions using the Gaussian function.

$$eff ( d ) = {e}^{- \frac{{( d - μ )}^{2}}{2 {σ}^{2}}}$$

Where
d is the dosage,
μ is the optimal dosage, and
σ determines the effectivity vs dosage response

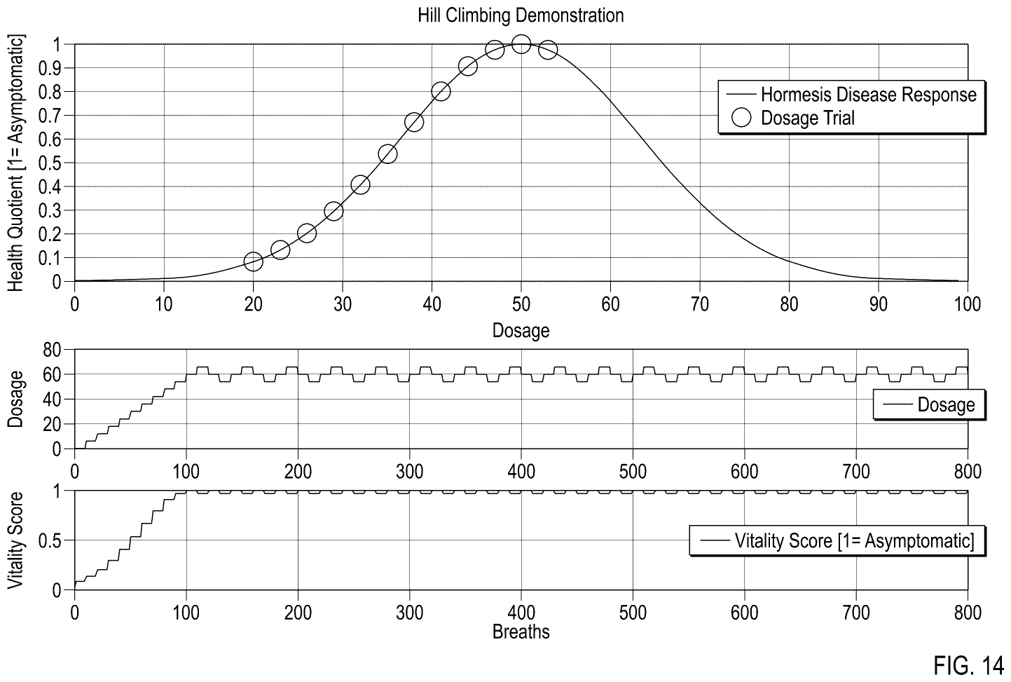

FIG. 14 shows the response of the machine to a hormesis response with and ideal dosage of 50 (μ=50) and a low starting dosage.

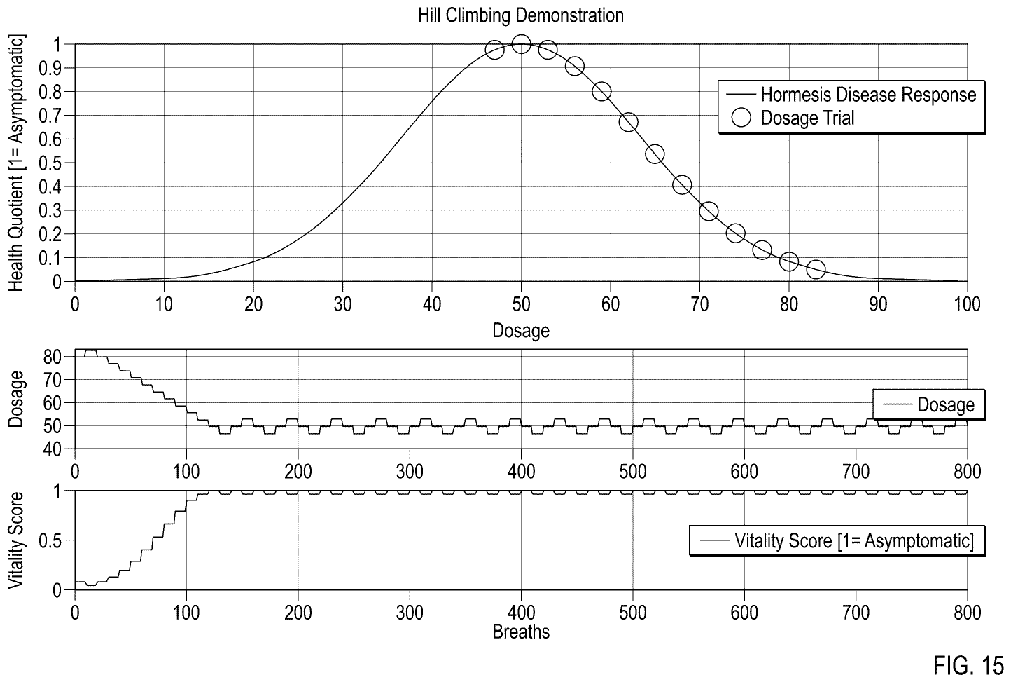

FIG. 15 shows the response to a hormesis response with an ideal dosage (μ=50) and a high starting dosage.

It can be seen that the machine performs well with both fundamental and hormesis shaped response situations. It is successful at finding the optimal dosage prescription as defined by the response curve.

### Stochastic Hill Climbing

For complex dosage/response relationships the hill climbing technique must be altered slightly. A stochastic method is chosen that during a break in therapy, the machine will jump to a random setting within range. The machine must also remember the best dosage and when the optimizer settles to an inferior dosage that presents as a local maximum but not a global maximum in the health score, it will automatically migrate to or near the previously found global maximum. Those familiar in the art will appreciate that the methods of hill climbing and stochastic optimization searches are all anticipated here to find the optimal health score using a constrained set of operational parameters (settings).

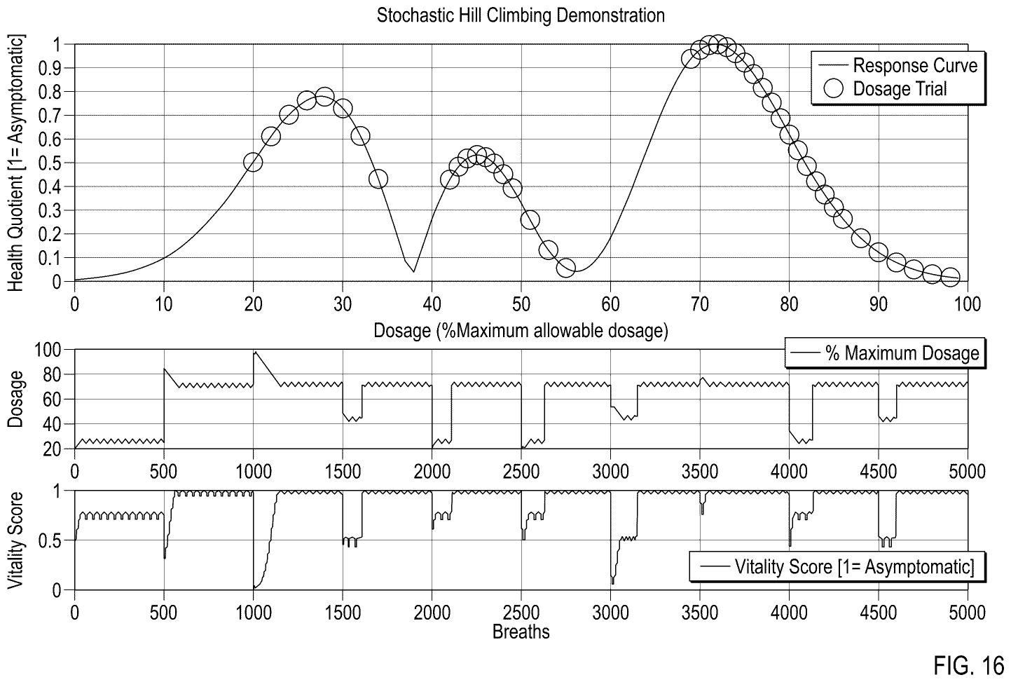

FIG. 16 shows the performance of the stochastic hill climber performing on a complex dose/response, starting at different dosages within range on different days. The machine will remember the best dosage when the device gets, “stuck” at an inferior dosage, it jumps to a previously found optimized setting.

Alternative Method of Determining when a Dosage is in the Ineffective Range (Circuit Breaker Method)

Alternatively to the stochastic method, the user may instruct the machine to automatically increase the dosage when the measurements are so far outside the range of vitality. An example of this could be when a patient with OSA is experiencing a frank apnea and no breaths are measured to evaluate a breathing parameter. The recognition of apnea may be programmed as a circuit breaker running outside the hill climbing method to bypass the trial and immediately increase or decrease the dosage as appropriate. For apnea the clinician can program the device to immediately increase the dosage of EPAP when the frank apnea is detected.

If the response is always to increase dosage, the circuit breaker method fails for a hormesis response, the stochastic method is preferred as a universal method rather than a programmed circuit breaker for dosages with a hermetic response.

Optimizing More than One Setting

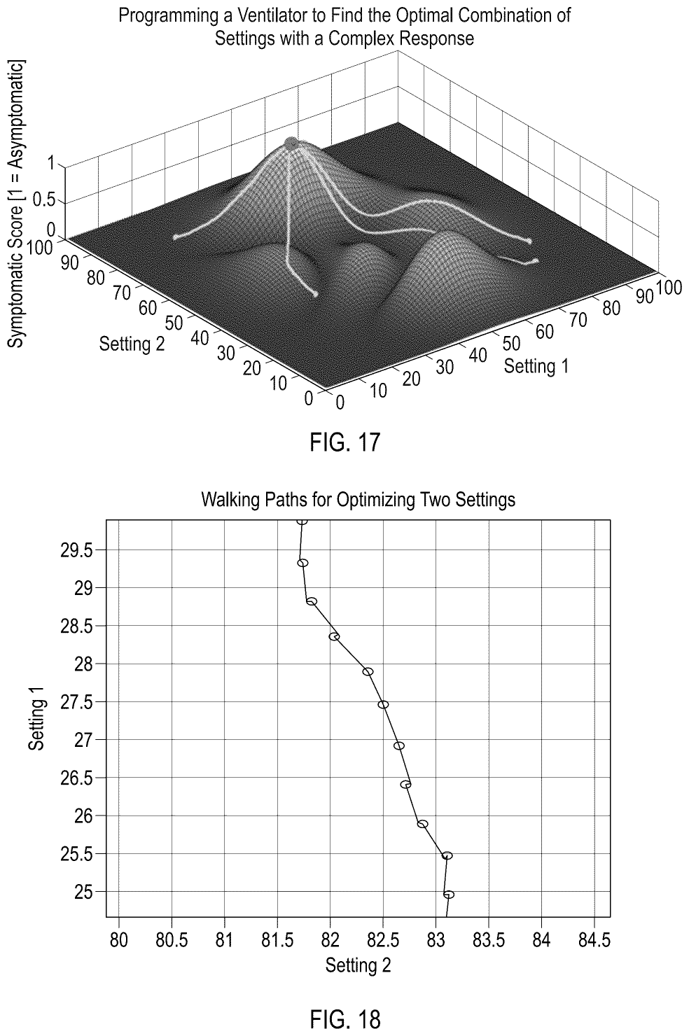

The hill climbing technique can be expanded to more than one dimension (See FIG. 17). The machine will follow the steepest gradient at a given prescription, by doing a n dimensional walk to discover the steepest gradient in the neighborhood of the current prescription. If we consider changing two settings together, the search pattern should approximate a circular walk around the current therapy to determine the direction of steepest ascent. If the setting dimension is three, a trial of parameters spherically positioned around the values of the current conditions are trialed and so on, up to as many dimensions as there are changing parameters. Here the machine follows that direction that provides the steepest ascent in health score compared to the current setting. The circular walk is repeated regularly to readjust the direction of therapy changes.

The method can be stochastic, randomly beginning at a combination of settings and remembering which settings provided the optimal settings when the machine “settles” to an inferior setting.

As illustrated in FIG. 17, following the steepest ascent, the machine finds the optimal settings to reduce symptoms (indicated by the red dot) on this complex surface. The machine begins at random setting combinations within the ranges to maximize the probability that the optimal settings will be eventually found.

FIG. 18 further illustrates the circular search patterns that are routinely performed to find the direction of the steepest ascent. Small changes are made to the current settings in a circular pattern, making measurements to determine the steepest ascent away from the current settings.

Higher dimensions, such as 3 settings, would require a spherical search. The geometry of the search would become more complex as the dimensionality is increased and the search pattern is a hyperplane in the higher dimensional space, but the basic concept is the same by taking the direction of gradient of steepest ascent.

### Exemplary Ventilator Programming for Auto-Adjustment of the Operational Settings

First, we consider for the patient under treatment, what is in the underlying chronic disease that requires a mechanical ventilator. Next, we consider a mode of ventilation suitable for treating this patient. We pick operational settings in this mode.

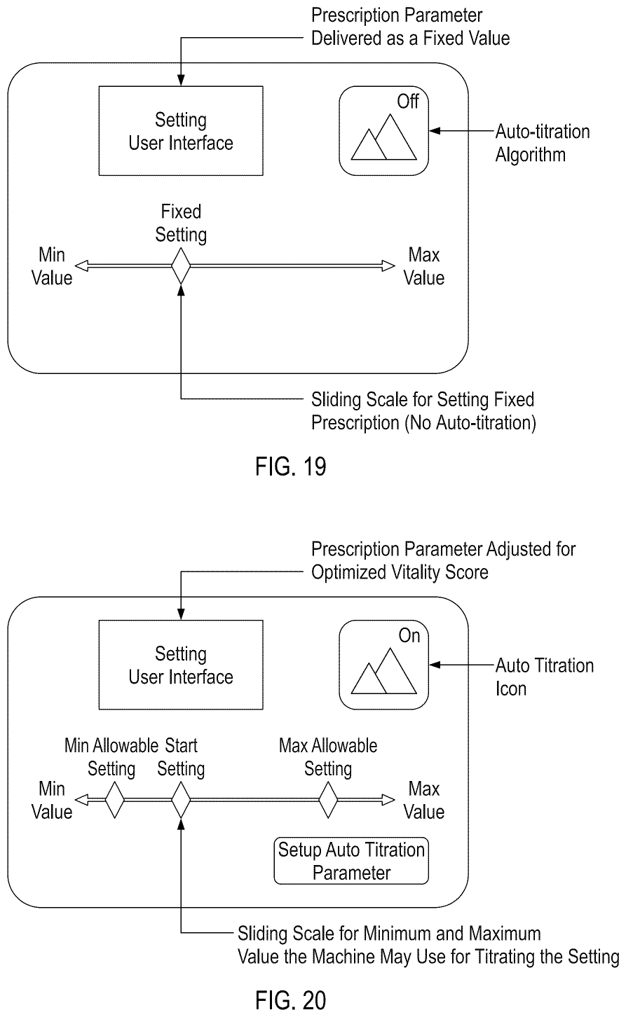

For settings that are to be fixed throughout the treatment, the clinician will describe a fixed setting. (For example, S/T mode with IPAP=20 cm H20) (See FIG. 19).

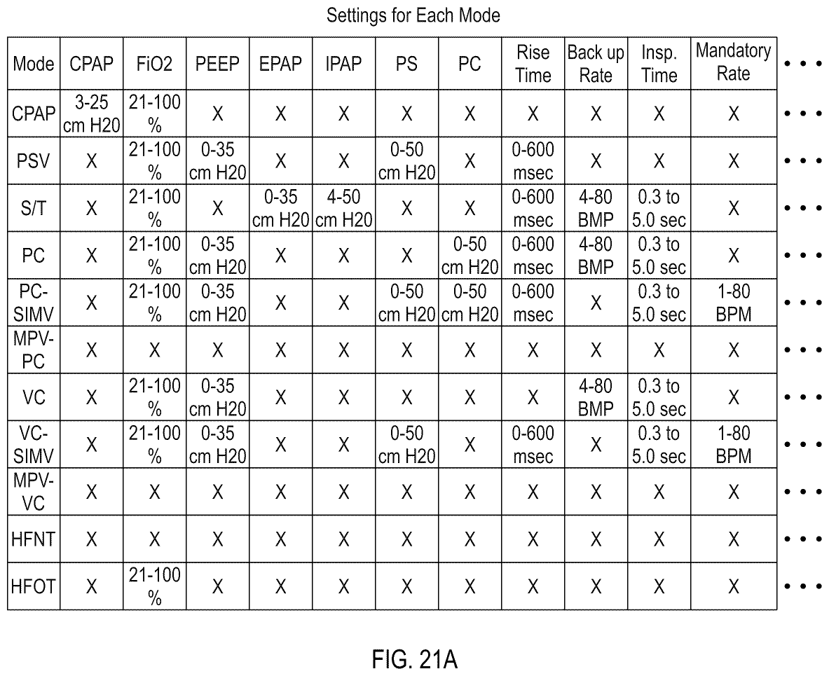

For settings that are to be auto titrated, this require a range of values from the minimum value to the maximum value. (For example, S/T mode with IPAP ranging from 15 to 25 cm H20) (See FIG. 20)).

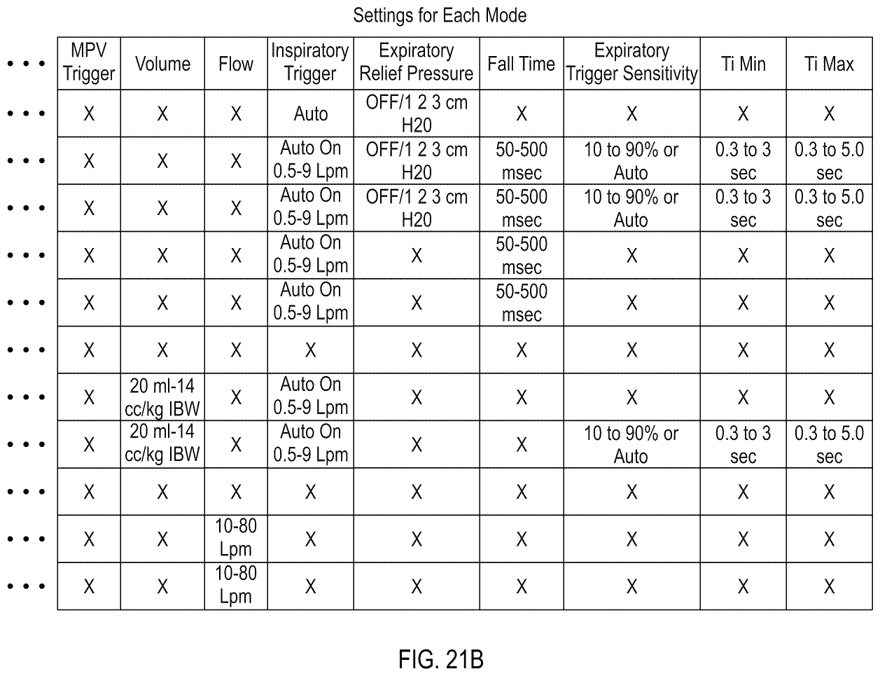

A full range of exemplary setting parameters can be seen in FIG. 21.

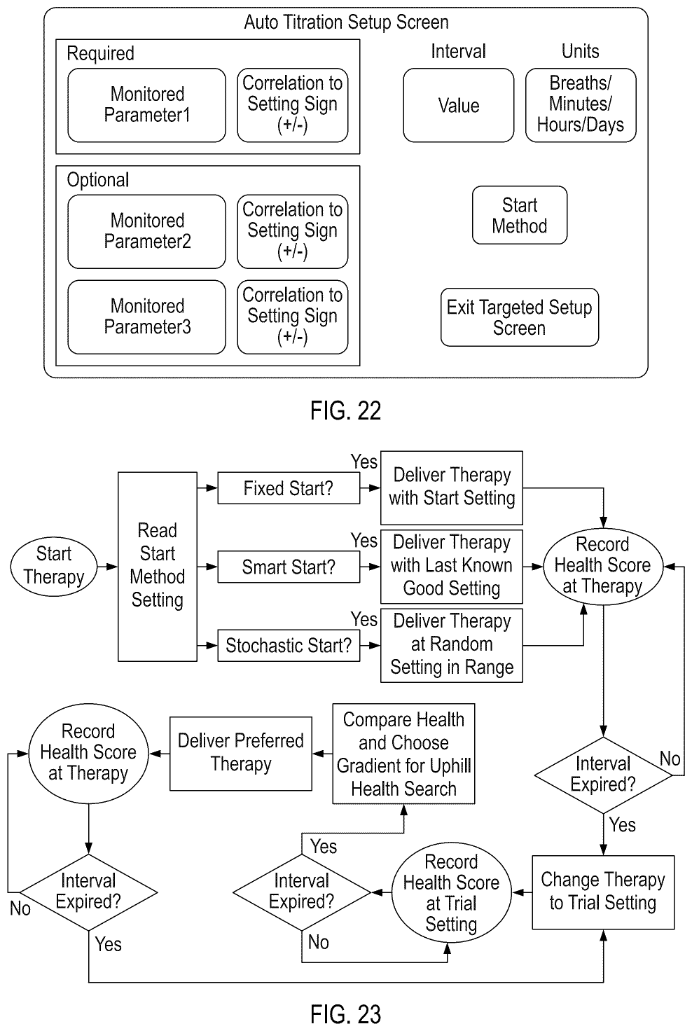

Next we chose one or more biometric readings or settings to evaluate the vitality of the patient at the given prescription (See FIG. 22).

Here we can decide for example, we wish to optimize IPAP, to minimize RR and CO₂.

Respiratory Rate and TcCO₂ (transcutaneous carbon dioxide) are negatively correlated to vitality. (Positively correlated to symptoms)

$$Health Score = \frac{{}^{‵} 1}{RR \cdot Tc CO 2}$$

Next, we choose an interval at which the algorithm runs. (For example, we chose 1 minute intervals, meaning that the Health or Vitality score will be taken as a mean, median or most recent value every 1 minute of therapy before the IPAP prescription is adjusted attempting to optimize this health score).

As will be described in more detail hereinbelow, the invention provides a novel, programmable ventilator system and methodology where the clinician may prescribe and individualize the selection of ranges of operational settings and monitoring of selected patient biometrics. The ventilator system includes an automated response system which seeks to optimize a Health or Vitality Score for the patient by systematically titrating selected operating settings within prescribed minimum and maximum ranges until the Vitality score stabilizes at a reading as close as possible to the maximum Vitality score. The prescribing clinician may define a single operational setting to be titrated or a combination of any one or more operational settings. The Vitality score is computed from monitored patient biometric sensor reading and/or any measured corresponding device parameter or calculated biometric marker to define the dynamic control of the ventilator system to automatically normalize the patient to the highest possible Vitality score.

A flow chart of operation is illustrated in FIG. 23.

In a first embodiment, the system is programmed with an initial “therapeutic” configuration and/or mode having a set operational mode and/or operational settings. For example, in the base therapy state, the patient may be receiving Continuous Positive Airway Pressure (CPAP) with particular operational parameters, such as pressure and FiO₂.

In accordance with the teachings of the present invention the system is further programmed with a managed auto-titration or adjustment protocol to periodically calculate a patient health score and then determine incremental adjustments of one or more settings within the prescribed minimum and maximum ranges which will advance (improve) the patient's health score towards the optimal or maximum possible health score.

The present invention overcomes the issues of inappropriate response time incurred due to remote monitoring and more specifically the issue of “no response” when caregivers are absent, or the patient is unable to intervene on his own.

There are no underlying algorithms contained within the device programming to make clinical assessments of the patient, and the device does not decide what an appropriate response could be. Biometric and operating conditions are measured, health scores are calculated, and a prescribed response interval is set based on the judgment of the physician attending to the specific patient who is treated. The system will simply only operate according to the direction of the programming clinician. There is full transparency of how, when, and why a setting will change.

An important distinction with Programmable Ventilation with auto-titration using hill climbing according to the present disclosure, is that modification or adjustment of the ventilator's operational parameters are solely controlled by the heath data signals that are already monitored and displayed by the device without providing any “diagnostic function” for analysis of the patient's biological condition and physiological state. The actions taken by the system are done automatically only according to the prescription of the expert clinician.

The control system adjusting the parameter or parameters is constrained to approved ranges ascribed and programmed for the device and the class of patient. In other words, the titration system will not adjust an operational parameter to a value that is not already allowed under the device's intended use or qualified range of settings (saved prescription). The procedures and techniques including the stability analysis, verification, validation will ensure compliance with the applicable safety standards and that all associated safety risks have been managed as far as possible in the design.

According to exemplary embodiments, a system and method are provided for dynamically controlling operation of a mechanical ventilator for automatic adjustment of one or more operational settings based on patient biometric data. A ventilator prescription includes a plurality of initial operational settings, minimum and maximum operational boundaries corresponding to each of the plurality of operational settings, parameters to be included in a health score by which the ventilator determines a current health score according to one or more biometric variables corresponding to the target person, an interval period for relating current operational settings of the ventilator system to a health score; and optimization system described by a hill climbing method by which the ventilator system identifies a modification of a predetermined one of the plurality operational settings to optimize the current health score, wherein the modification of the predetermined one of said plurality operational settings falls within the minimum and maximum operational setting boundaries. The ventilator system operates according to the initial configuration, monitors patient biometric variables, periodically determines a current health score, tracks in memory, the current health score related to the variable operational settings, navigates to the operational configuration within the boundaries that optimizes the health score, continually generates modifications of a predetermined operational settings (increase or decrease) in setting (dosage) to advance the current health score towards the changing optimal health score (hill climbing algorithm) associated with the patient disease, continually reconfigures the ventilator system according to the generated modification, and applies the best operational setting based on the health score.

In some embodiments, the initial configuration may comprise one or more predetermined operational settings and/or operating modes which include but are not limited to the following: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

The operational settings associated with these modes include but are not limited to: Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (AP). In many instances the base mode and intervention mode may be the same, but the intervention mode may include one of more differences in operation parameters that are all pre-determined by the prescribing clinician to affect positive outcomes when the patient is in need.

In some embodiments, the biometric variables may include, but are not limited to the following: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

In some embodiments, the biometric variables may further include calculated biomarkers such as Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (AP), Mechanical energy, Mechanical power and intensity, and Pressure-time product per minute (PTP).

In some embodiments, the optimization formulae identifies modifications of two or more of operational settings to optimize the current health score.

In some embodiments, the optimization formulae identifies a plateau condition of decreasing improvement to the current health score and maintains current operational settings in a steady state condition.

Specifically, an exemplary method for dynamically controlling the operation of a ventilator system in providing mechanical ventilation to a target person comprises the steps of:
accessing a prescription for automatic control of the ventilator system in providing mechanical ventilation to the target person, the prescription comprising:
an initial configuration comprising
   a plurality of initial operational settings of the ventilator system to provide an initial mechanical ventilation to the target person,
   minimum and maximum operational boundaries corresponding to each of the plurality of operational settings,
   a health score formulae representing a quantitative measure of response by which the ventilator determines a current health score according to one or more biometric variables corresponding to the target person;
   an interval setting defining an interval period for evaluating a current health score of the target person; and
   a selected optimization method by which the ventilator system systematically trials modifications of one or more of said plurality operational settings to improve the current health score.

The method further comprises the steps of:
configuring the ventilator system according to the initial configuration;
operating the ventilator system according to the initial configuration, and during operation of the ventilator system, periodically according to the interval setting,
   systematically trialing modifications to one or more of the plurality of operational settings according to the selected optimization protocol;
   determining an updated health score for the target person corresponding to the trialed operational settings;
   tracking in a memory, the current health score, current operational settings, updated health score and trialed operational settings; and
   upon determining that the updated health score is higher than the current health score,
   reconfiguring the ventilator system according to the trialed operational settings; and
   saving in memory the trialed operational settings as the current operational settings.

In the proposed method, the generated modification may be an increase in the predetermined operational setting (increased dosage).

In the proposed method, the generated modification may be a decrease in the predetermined operational setting (decreased dosage).

In the proposed method, the prescription may include titrating more than one operational setting.

The modification conditions are not the machine's clinical assessment that the initial parameter setting is no longer optimal, but that combination of respiratory (biometric) measurements which the clinician has deemed the modification condition describing when the patient requires a modification to optimize the health score, and or when the patient no longer requires the modification because the health score is optimal.

In some embodiments, the modification parameters may be further based on a correlation of the current condition score to the configuration of the ventilator system.

In some embodiments, the initial configuration may comprise one or more predetermined operational parameters and/or operating modes which include but are not limited to the following: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

The operational parameters associated with these modes include but are not limited to: Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (AP) and combinations of the modes noted and the operational parameters set forth above.

In some embodiments, the biometric sensor readings may include, but are not limited to the following: heart rate, respiratory rate, blood pressure, Oxygen Saturation (SpO₂) and End-Tidal Carbon Dioxide (ETCO₂).

In some embodiments, the biomarker calculators are selected from the group consisting of: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (AP), Mechanical energy, Mechanical power and intensity, and Pressure-time product per minute (PTP). Calculation of the noted biomarkers is generally known in the art and will not be further described.

Once initiated in auto-titration mode, the system operates according to the flow diagram as illustrated in FIG. 10 delivering the initial ventilation configuration according to the initial mode and settings.

The system memory 32 may allow more than one set of prescriptions to be stored along with respective operational settings.

In the present embodiments, the system seeks to achieve a highest possible Vitality Score without requiring an expert to manually detect the condition and the need to manually change the prescription and operating parameters.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A method for dynamically controlling the operation of a ventilator system in providing mechanical ventilation to a target person, comprising:
accessing a prescription for automatic control of the ventilator system in providing mechanical ventilation to the target person, the prescription comprising:
an initial configuration comprising
a plurality of initial operational settings of the ventilator system to provide an initial mechanical ventilation to the target person,
minimum and maximum operational boundaries corresponding to each of the plurality of operational settings,
a health score formulae representing a quantitative measure of response by which the ventilator determines a current health score according to one or more biometric variables corresponding to the target person;
an interval setting defining an interval period for evaluating a current health score of the target person; and
a selected optimization method by which the ventilator system systematically trials modifications of one or more of said plurality operational settings to improve the current health score,
configuring the ventilator system according to the initial configuration;
operating the ventilator system according to the initial configuration;
determining a current health score according to the initial configuration;
during operation of the ventilator system, periodically according to the interval setting,
systematically trialing modifications to one or more of the plurality of operational settings according to the selected optimization protocol;
determining an updated health score for the target person corresponding to the trialed operational settings;
tracking in a memory, the current health score, current operational settings, updated health score and trialed operational settings; and
upon determining that the updated health score is higher than the current health score,
reconfiguring the ventilator system according to the trialed operational settings; and
saving in memory the trialed operational settings as the current operational settings.

2. The method of claim 1, wherein the generated modification is an increase in the predetermined operational setting.

3. The method of claim 1 wherein the generated modification is a decrease in the predetermined operational setting.

4. The method of claim 1, wherein the initial configuration comprises one or more predetermined operational settings and/or operating modes selected from the group consisting of: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T); Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP) and combinations thereof.

5. The method of claim 1 wherein the one or more biometric variables are selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), Respiratory Rate Oxygenation (Rox) and combinations thereof.

6. The method of claim 4 wherein the one or more biometric variables are selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), Respiratory Rate Oxygenation (Rox) and combinations thereof.

7. The method of claim 5 wherein one or more biometric variables are further selected from the group consisting of: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (AP), Mechanical energy, Mechanical power and intensity, Pressure-time product per minute (PTP), and combinations thereof.

8. The method of claim 6 wherein one or more biometric variables are further selected from the group consisting of: Peak inspiratory pressure (PIP), Peak pressure, Inspiratory time, Inspiratory-to-expiratory ratio, Time of pause, Trigger sensitivity, Support pressure, Expiratory trigger sensitivity, Plateau pressure (Pplat), Transpulmonary pressure, Transpulmonary driving pressure (ΔP), Mechanical energy, Mechanical power and intensity, Pressure-time product per minute (PTP), and combinations thereof.

9. The method of claim 1 wherein the optimization method is selected from the group consisting of: a hill climbing method, a stochastic hill climbing method, two-dimensional hill climbing method, and an n-dimensional hill climbing method, each method optionally including a circuit breaker method.

10. The method of claim 4 wherein the optimization method is selected from the group consisting of: a hill climbing method, a stochastic hill climbing method, two-dimensional hill climbing method, and an n-dimensional hill climbing method, each method optionally including a circuit breaker method.

11. The method of claim 4 wherein the optimization method is selected from the group consisting of: a hill climbing method, a stochastic hill climbing method, two-dimensional hill climbing method, and an n-dimensional hill climbing method, each method optionally including a circuit breaker method.

12. The method of claim 5 wherein the optimization method is selected from the group consisting of: a hill climbing method, a stochastic hill climbing method, two-dimensional hill climbing method, and an n-dimensional hill climbing method, each method optionally including a circuit breaker method.

13. The method of claim 1 wherein the optimization formulae identifies a plateau condition of decreasing improvement to the current health score and maintains current operational settings in a steady state condition.

14. The method of claim 4 wherein the optimization formulae identifies a plateau condition of decreasing improvement to the current health score and maintains current operational settings in a steady state condition.

15. A dynamically configurable ventilator system for providing mechanical ventilation to a target person according to a prescription, configured to operate in accordance with the method of claim 1.
